(19) United States
(12) Patent Application Publication
(10) Pub. No.: US 2026/0276642 A1
(43) Pub. Date: Sep. 17, 2026

(54) METHOD AND KIT FOR THE DIAGNOSIS OF ORAL CARCINOMA

(71) Applicants: Riccardo MOFFA, Monaco (MC); Angelo Simone PARODI, Monaco (MC); Emanuela MARCENARO, Genova (IT)

(72) Inventors: Riccardo MOFFA, Monaco (MC); Angelo Simone PARODI, Monaco (MC); Emanuela MARCENARO, Genova (IT)

(21) Appl. No.: 19/167,412

(22) PCT Filed: Mar. 21, 2024
(86) PCT No.: PCT/IB2024/052732

§ 371 Date: Sep. 22, 2025

(30) Foreign Application Priority Data
Mar. 21, 2023 (IT) 102023000005358

(51) Int. Cl. G01N 33/575; G01N 21/64; G06V 10/28; G06V 20/69

(52) U.S. Cl. CPC G01N 33/57557; G01N 21/6486; G06V 10/28; G06V 20/69; G01N 2333/4703; G01N 2333/70532; G01N 2333/71; G06V 2201/03

(57) ABSTRACT

A method and kit are used to analyze the protein levels of EGFR, p53, B7H6, PdL1 in a sample from a subject and estimate the presence of oral squamous cell carcinoma in the subject. The method and kit compare the protein levels in the sample with levels of corresponding proteins in samples isolated from control individuals and/or with levels recorded in reference databases.

### CROSS REFERENCE TO RELATED APPLICATIONS

This application is a national stage entry of International Application No. PCT/IB 2024/052732, filed on Mar. 21, 2024, which is based upon and claims foreign priority to Italian Patent Application No. 102023000005358, filed on Mar. 21, 2023, the entire contents of which are incorporated herein by reference.

### TECHNICAL FIELD

The present invention relates to a method performed in vitro for determining the presence of squamous cell carcinoma in the oral cavity, preferably at an early stage, based on measuring the levels of a specific series of protein markers.

### BACKGROUND

More than 90% of oral cancers are oral squamous cell carcinomas (OSCC).

Oral squamous cell carcinoma (OSCC) is the sixth most common cancer worldwide. Oral cancer has an average five-year survival rate of about 60% (1). Treatments for advanced stage oral cancer require mutilating interventions and the evolution of the disease leads to a very low quality of life. To reduce mortality and morbidity, oral cancer should be detected at a very early stage in order to improve the efficacy of available therapies.

To date, biopsy is the most effective method of collecting tissue useful for diagnosis. It requires surgery and does not allow early diagnosis because it is performed on lesions that are already visible on clinical examination. To date, beyond the conventional oral clinical examination, with or without the aid of magnifying optics and fluorescent systems, there are no scientifically approved systems capable of detecting a lesion in the early stages of tumor transformation and the diagnostic gold standard remains the biopsy and immunohistochemical staining.

This method has some limitations. It is semi-quantitative and the absolute abundance of the target cannot be reliably determined. In addition, the tissue is highly processed and may lead to the loss of information about the natural state.

In other medicine fields, such as gynecology, early detection of cervical cancer through cytobrush biopsy, PAP test and HPV test has proved highly effective in reducing mortality, morbidity and costs for the community. The oral cytobrush (2-6) is able to collect tissue particles, cells and a small amount of saliva at the same time.

The ability to detect molecules with the cytobrush from patients with this type of tumor has recently been suggested, however, no single biomolecule has been shown to meet the real-world requirement for high accuracy in identifying early disease onset, suggesting the need to develop a non-invasive, fast and effective method of diagnosis in classifying and identifying suspicious oral lesions.

### SUMMARY

Method and kit to analyze the protein levels of EGFR, p53, B7H6, PdL1 in a sample from a subject and to estimate the presence of oral squamous cell carcinoma in the subject.

### BRIEF DESCRIPTION OF THE DRAWINGS

The present invention is now described by way of non-limiting example by means of the following drawings.

FIG. 1: The names of the different markers are shown in the column (the first triplet EGFR, p53, Ki67, control; the second triplet B7H6, PDL1, HLAE, control); in the row the times of the different chemiluminescence acquisitions (t1, t2, t3). The numbers represent the chemiluminescence values found. The values presenting a FM between 1.3 and 1.49 compared to the control are marked in yellow, the values presenting a FM greater than 1.49 compared to the control are marked in red.

FIG. 2: The calculation of the FMs for each marker at the three different times is shown only if this is greater than 1. For each marker that has FMs greater than 1, at the bottom of the corresponding column, the sum of the FM of the three strokes is reported.

FIG. 3: The table shows for each topological area considered (from top to bottom: tumor center (FIG. 3A), healthy (FIG. 3B), margin (FIG. 3C)) the sum of the FMs of each marker for the patient in example; below is the sum of the FMs of all the markers for the topological area (A, B, C); finally the sum of only the markers EGFR, PDL1 and B7H6. Table C shows the FM signal intensity delta, i.e. the difference between the sum of the FM neoplastic tissue and the margin (3,2). The corresponding graphs show, by means of a pie chart, the expression percentage of each marker with respect to the total intensity of the signal, given by the sum of all the FMs, for the considered area. The healthy tissue area has FM values equal to zero for all markers.

FIG. 4: The names of the different markers are shown in the column (the first triplet EGFR, p53, Ki67, control; the second triplet B7H6, PDL1, HLAE, control); in the row the times of the different chemiluminescence acquisitions (t1, t2, t3). The numbers represent the chemiluminescence values found. The values showing a FM between 1.3 and 1.49 compared to the control are marked in light grey, the values showing a FM greater than 1.49 compared to the control are marked in dark grey.

FIG. 5: The calculation of the FMs is shown for each marker at the three different times, only if this is greater than 1. For each marker that has FMs greater than 1, the sum of the FM of the three times is reported at the bottom of the corresponding column.

FIG. 6: Biopsy samples. The table shows for each topological area considered, from top to bottom: tumor center (A), healthy (B), margin (C) the sum of the FMs of each marker for the patient in example; below is the sum of the FMs of all markers and below the sum of only the markers EGFR, PDL1 and B7H6. Finally, the FM signal intensity delta, i.e. the difference between the FM sum of neoplastic tissue and margin (0) and healthy tissue (0). The graph shows, by means of a pie chart, the expression percentage of each marker with respect to the total intensity of the signal, given by the sum of all the FMs, for the considered area. The area of healthy tissue has values only for Ki 67 which therefore appears to be 100% of the signal.

FIG. 7: perspective exploded view in longitudinal section of a disposable kit according to the present invention illustrating 4 lines.

FIG. 8: enlarged perspective view of FIG. 7.

FIG. 9: sectioned perspective view of a component of the kit of the present invention assembled in use configuration.

### DETAILED DESCRIPTION OF THE EMBODIMENTS

In order to overcome the obstacles of the prior art, the inventors searched for, identified and validated protein markers that could be used for a non-invasive, fast and effective screening test in classifying and identifying oral squamous cell carcinomas (OSCC) and identifying suspected oral lesions with morphologically altered tissue as precancerous. By way of non-limiting example, such oral lesions can be for instance leukoplakia, erythroplakia, palatal smoking lesion, oral lichen planus, oral submucous fibrosis (SMF) and discoid lupus erythematosus.

The object of the present invention was therefore to select suitable markers to be detected in samples obtained preferably with non-invasive procedures by means of a rapid ELISA (enzyme-bound immunosorbent assay) technique, preferably executable by the operators following the obtaining of the sample and verifying whether the detection of these biomarkers could discriminate OSCC lesions or precancerous lesions from the surrounding healthy tissue and create a kit usable in an in vitro diagnosis method of this pathology.

In a preferred embodiment, the technique is an ELISA technique with signal detection of the colorimetric type or with signal detection based on luminescence.

By way of non-limiting example, both portions of biopsies obtained from patients and samples obtained with non-invasive procedures are considered as optimal samples, i.e. samples obtained from oral smears performed with sterile swabs for cytological sampling, for instance cytobrush, saliva samples and in general samples containing epithelial cells from the oral cavity.

The inventors have selected six biomarkers, of protein type, chosen among different groups of markers:
markers already used in immunohistochemical practice for the diagnosis of OSCC,
markers known to be present or overexpressed in transforming cells
markers identified as possible molecular targets in immune checkpoint blockade therapies.

The inventors initially evaluated the expression levels of several selected markers, EGFR (epithelial grow factor receptor) sequence preferably corresponding to the sequence available in the NCBI database with accession number NM-005228.3; AR (androgen receptor) sequence preferably corresponding to the sequence available in the NCBI database under accession number NM-000044.4; ER (estrogen receptor) sequence preferably corresponding to the sequence available in the NCBI database under accession number NP-001428.1; PDL1 (programmed death ligand 1) sequence preferably corresponding to the sequence available in the NCBI database with accession number Q9NZQ7.1; B7H6 sequence preferably corresponding to the sequence available in the NCBI database with accession number Q68D85; Ki67 sequence preferably corresponding to the sequence available in the NCBI database with accession number P46013.2; HLAE (class 1 Histocompatibility antigen) sequence preferably corresponding to the sequence available in the NCBI database with accession number P13747.4; P16INK4A sequence preferably corresponding to the sequence available in the NCBI database with accession number P42771.2; p53 sequence preferably corresponding to the sequence available in the NCBI database under entry number P04637.4.

The inventors initially performed the evaluation of the expression of these associated markers in healthy or with general inflammation of the oral cavity (leukoplakia, lichen, etc.) volunteer patients. All the markers are of the protein type and were analyzed by ELISA technique, in particular a quantitative sandwich ELISA technique was used for the analyses.

The samples were collected by cytobrush after having rinsed the patient's mouth with saline and arrived in the laboratory in a closed refrigerated container. At the time of the analysis (within 72 h) a lysis solution was added and each marker was evaluated by binding to an antibody sandwich, preferably a rabbit polyclonal antibody anchored to a PVDF membrane and a specific mouse monoclonal antibody.

Each sample subjected to lysis was incubated with a PVDF membrane previously loaded with the corresponding polyclonal antibody for the recognition of the marker of interest. A sandwich ELISA test was performed with chemiluminescent signal detection using Luminol reagent (ECL, Amersham Cytiva). The light intensity emitted by each specific immunological reaction was detected and analyzed with dedicated software (fiji app software).

This preliminary study demonstrated how some tumor markers were not expressed in healthy patients and not expressed or poorly expressed in patients with inflammation of the oral cavity, demonstrating their specificity for exclusive situations of cancer as the literature suggests. This is the case of the markers EGFR, p53, Ki67, B7H6, PDL1, HLAE. Other markers (AR, ER, p16), on the other hand, showed high expressions in healthy patients or at least greater than in patients with oral cavity infections, they generally did not show specificity for inflammatory conditions, and were therefore excluded from more specific studies conducted later.

The following were therefore selected for in-depth studies:
EGFR-Epidermal growth factor receptor (EGFR) is a member of the ErbB tyrosine kinase (TK) receptor family.
p53—Cellular tumor antigen p53, acts as a tumor suppressor in many types of tumor; induces growth arrest or apoptosis depending on the physiological circumstances and cell type.
Ki67, a factor required to keep individual mitotic chromosomes dispersed in the cytoplasm after the nuclear envelope has been disassembled.
PD-L1—Programmed cell death ligand 1, a factor that plays a critical role in the induction and maintenance of autoimmune tolerance.
HLA-E, is a class 1 nonclassical MHC ubiquitously expressed in hematopoietic cells and is sensitive to inflammatory signals. HLA-E binds the CD94/NKG2A heterodimeric complex.
B7-H6—is a chimeric antigen expressed by some primary tumors and is recognized by the NKp30 receptor expressed on T (NK) cells.

The markers were selected to carry out an in-depth study in patients with oral cavity cancer at different stages of the disease (ten patients with tumors from stage t1 to t4), and were analyzed by a Quantitative ELISA technique using an automated device for signal detection. For each patient, three different samples were taken with cytobrush in three topological areas of the mouth: tumor center area, tumor margin and healthy tissue. Samples with light intensity having a control threshold value lower than 0.2 or greater than 20 were excluded from the results, this value is a dimensionless number given by the average light intensity of the area considered by the software; for each pixel of the area there is a BLUE value from 0 to 85 and the software averages all the pixels; samples degraded due to heat or transport problems were excluded.

The light intensity developed by the immunological reaction, represented by a dimensionless number calculated with specific software as detailed in the examples, was evaluated for each marker and for the control. For each test, the intensities at three different times (indicated with T1, T2, T3) were developed to analyze the trend of the signal over time. When a marker has an intensity value greater than the control in all three times, it is considered an expressed marker and its FM (multiplication factor) is calculated; the value of the multiplication factor is given by the ratio between the intensity value of the channel under examination at time t and that of the control at the same time.

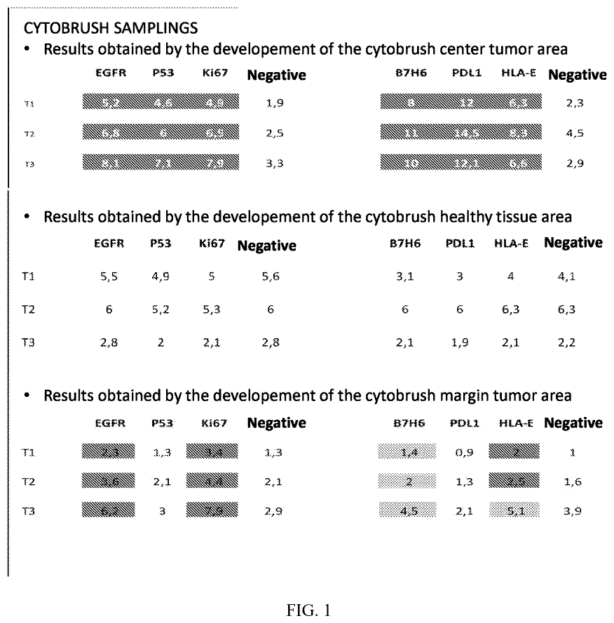

Following internal control tests, to an FM value lower than 1.2 a nonsignificance signal (the corresponding signal value is not colored in FIG. 1), to an FM value between 1.3 and 1.49, a moderate signal value (the corresponding signal value is marked in light gray in FIG. 1) and to an FM value greater than 1.49, a neat signal (the corresponding signal value is marked in dark gray in FIG. 1) were assigned.

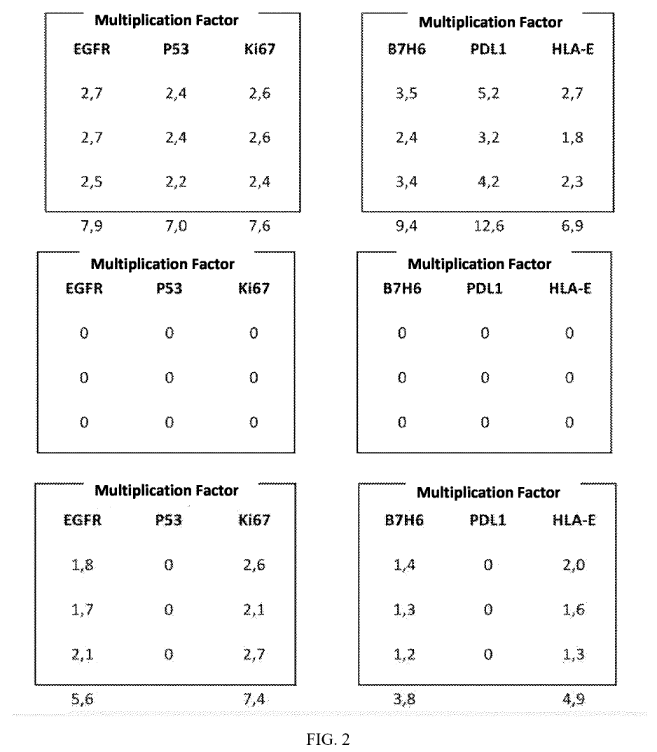

For each marker, the sum of the FMs for the three times considered was then calculated and reported in the following FIG. 2.

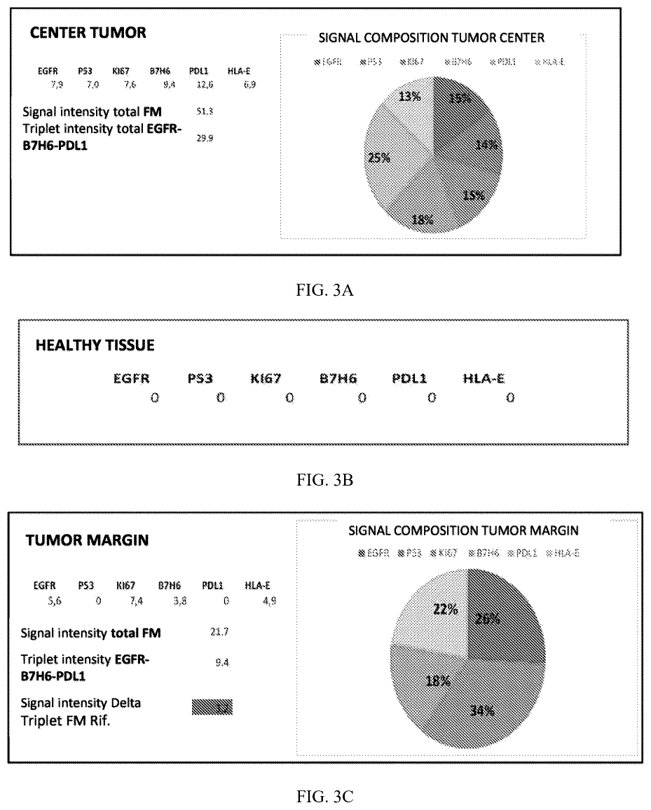

The sum of the single channels at the three times, previously calculated, has been reported in FIG. 3 and used to calculate the intensity of the total signal given by the single sums of all 6 markers analyzed (F.M. EGFR+p53+Ki67+B7H6+PDL1+HLAE). This operation was performed for each topological area considered (tumor center, table panel A, healthy tissue panel B, margin panel C) and the result is represented in the various graphs in the form of a percentage.

In addition to the total signal intensity, in FIGS. 3 A and C a further analysis was made taking into consideration only the signal intensity of the triplet EGFR, B7H6 and PDL1 as they are considered the most significant markers for a differential analysis of oral cavity cancer.

Finally, the differential delta (numerical ratio) between the signal intensity of the EGFR-B7H6-PDL1triplet in the tumor center and the signal intensity of the same reference triplet in the margin was reported in panel C to demonstrate how it is more expressed in the tumor center sample.

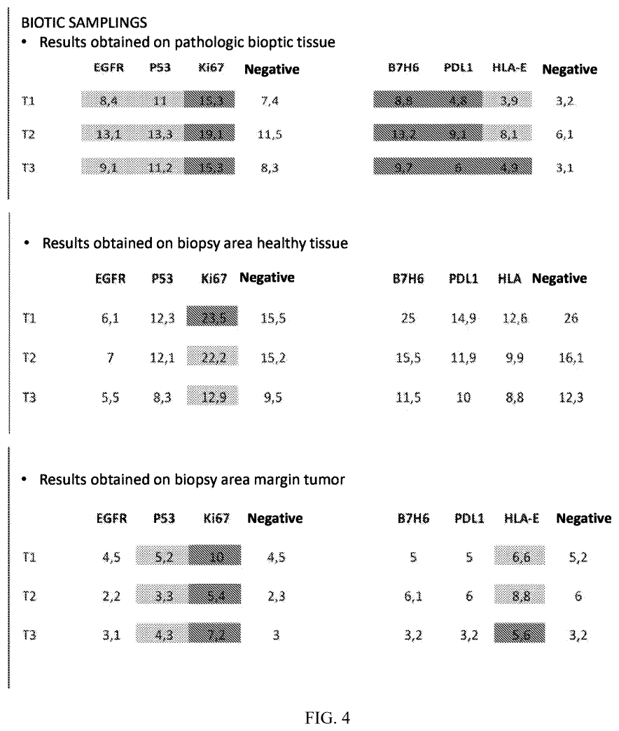

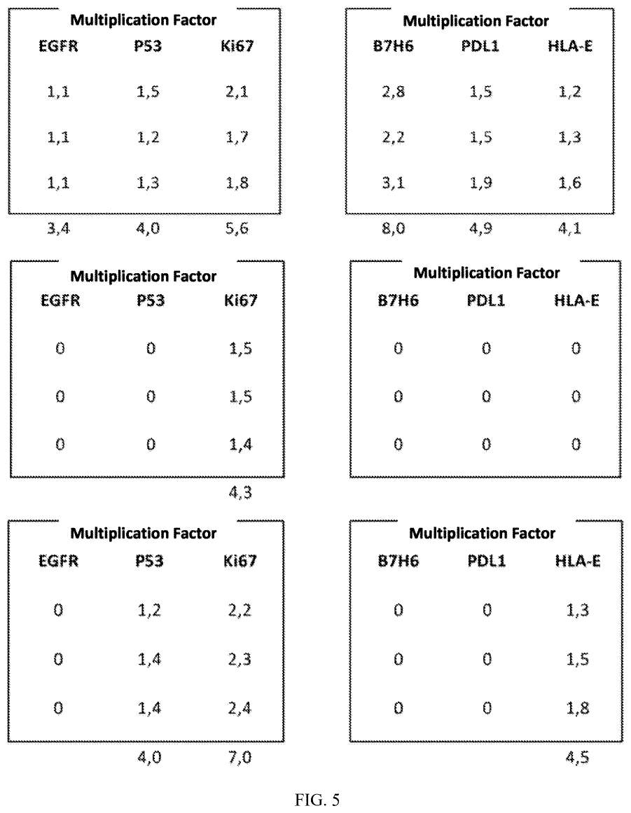

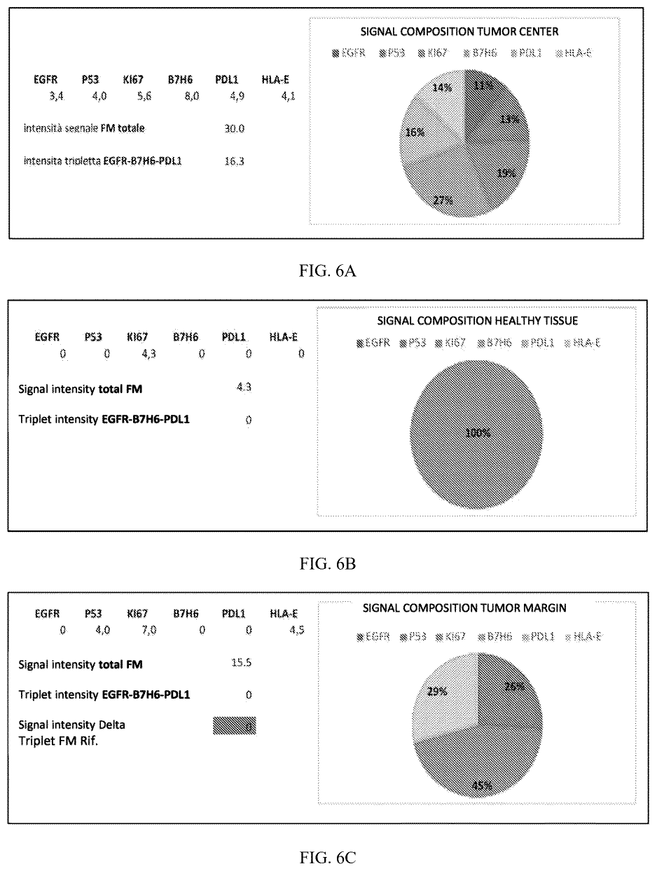

In order to verify the results obtained, and since additional material to be examined in the form of a small biopsy fragment was available for some of the patients with oral cavity cancer already analyzed by brush biopsy, the inventors performed the analysis by means of the ELISA technique also for this different provided material. The results obtained, in terms of expressed markers, were then compared to the same patient, for the same topological area considered, with respect to the brush biopsy. The sample was analyzed by the same sandwich ELISA technique on the same markers obtained with brush biopsy. The qualitative and quantitative analyzes in this stage and the parameters considered are the same described in the paragraph relating to the tests carried out on the ten patients with oral cancer analyzed with brush biopsy. The results are shown in FIGS. 4, 5, 6. see above.

These studies conducted on the markers EGFR, p53, Ki67 (first triplet of markers in this order, represented in FIG. 1, plus the control on the last channel) and B7H6, PDL1, HLAE (second triplet represented in FIG. 1, plus the control) , in neoplastic tissue, margin and healthy tissue, were evaluated by paying attention to which markers had an intensity value greater than the control and, if so, what was the corresponding FM.

All the samples taken from the center of the tumor showed the presence of a chemiluminescent signal superior to the control for all six markers of interest, defining a phenotype characterized by the overexpression of the six markers, in accordance with the histological examination and the neoplastic report.

All the samples taken from healthy tissue showed the general absence of chemiluminescent signal for the six markers of interest, defining a phenotype characterized by the absence of expression of all six markers, in general with a maximum of 2 markers out of 6, in agreement with clinical indication on healthy tissue.

Samples on the margins of the tumor lesion showed the presence of chemiluminescent signal from 4 to 6 markers, defining a phenotype in accordance with clinical sampling distal to the tumor centre, both for the quality of the turning on and for the reduced intensity of the signal.

The sum of the FMs of each marker expressed in digits in FIG. 3, showed values that tended to be greater in the lesion center areas (for instance the EGFR marker with a value of 7.9 in the example in FIG. 3) compared to both the margin (EGFR 5.6 in the example in FIG. 3) and to healthy tissue (0); in particular, the sum of the FMs of all six markers was clearly greater in the samples taken from the center of the lesion (51.3 in the example in FIG. 3) than both at the margin (21.7 in the example in FIG. 3) and in healthy tissue (0).

Considering only the triplet of markers EGFR, B7H6 and PDL1, the sum of the FMs of the three markers is more accentuated than the sum of all six markers in the tumor tissue (29.9 in the example in FIG. 3) with respect to the margin (9.4) and to healthy tissue (0); showing neat intensity deltas (3.2 with respect to the margin, in the example in FIG. 3).

The study carried out on cancer samples analyzed by biopsies showed results comparable to the same study carried out on cytobrush samples, i.e. a pathological sample phenotype represented by the expression of all six markers in analysis; a marginal sample phenotype represented by the expression of four, five or in very few cases six markers analyzed and a healthy tissue phenotype represented by the general non-expression of the six markers or with a maximum of two markers expressed. The example in the figure shows a healthy sample with only the Ki67 marker expressed, a marginal sample with 4 overexpressed markers and a tumor tissue with overexpression of all six markers. The total FM intensity for the tumor tissue has a value of 30, in the margin it has a value of 15.5 and in the healthy one a value of 4.3. The differential delta is therefore zero, both between tumor tissue and healthy tissue and tumor tissue and marginal tissue. In general, the results obtained between healthy, marginal and pathological tissue in cancer samples taken by cytobrush found a high correspondence also for the biopsy fragments analyzed in the same topological areas. In the light of the data obtained after analyzing the results of the study relating to the six markers and after suggestions acquired from the scientific literature, it was decided to exclude two further markers. The excluded markers are Ki67 and HLAE. Ki67 as regards the patients subjected to the study, is very often present in healthy tissues as well as in the center of the lesion, not allowing a differential analysis between the different samples if only the markers in question are considered. HLA-E is suggested by the literature as a more suitable marker for a therapeutic action against cancer rather than a diagnostic marker. HLA-E, as well as PDL1, is a ligand with an inhibitory action on the immune response, normally present at low expressions in tissue cells; it is bound by the CD94/NKG2A receptor complex present on the NK cells of the immune system which recognizes them as “self” and does not attack them. Some tumors put into practice a defense system against NK, overexpressing their HLA-E so as not to be recognized as foreign cells. Cancer treatments based on the role of HLA-E are being developed in recent times; in fact, the use of specific monoclonal antibodies is increasingly common, which act negatively on the binding between HLA-E expressed by tumor cells and its receptors on NK, thus avoiding the binding with consequent destruction of the tumor cell. An example of a monoclonal antibody used is Monalizumab, whose efficacy is being verified by numerous studies and trials. However, not all tumors adopt this mechanism, so some dysplasias do not have the HLA-E ligand; this requires a rethinking of this ligand as a diagnostic marker of pathological conditions. With these premises, four markers were selected as markers for the diagnosis of OSCC: EGFR, P53, B7H6 and PDL1, which have demonstrated an effective diagnostic role and are supported by scientific literature as well as already used in common laboratory practices for diagnosis of neoplasms.

These data suggest that this fast and non-invasive phenotyping technique is able to support clinical practice, also providing precise indications on the characteristics of the oral lesion.

The present invention therefore refers to a method-implemented in vitro-for the diagnosis and/or prognosis, preferably at an early stage, of neoplasms of the oral cavity, preferably of oral squamous cell carcinomas. The method according to the invention is based on the selection of protein markers suitable for the purpose and on the measurement of the levels of the same in isolated samples, from biopsies of tissues of the oral cavity or from cellular samples of the oral cavity performed for instance by means of a cytobrush and relative comparison of said levels with the levels of the same marker in non-tumor samples isolated from healthy subjects. Furthermore, the present invention also refers to a kit for implementing the above method.

The object of the present invention is therefore a method for the diagnosis and prognosis of an oral squamous cell carcinoma in vitro comprising the following basic steps:
providing an isolated sample from said individual, analyzing the protein levels of EGFR, p53, B7H6, PdL1.
comparing said levels with the levels of the corresponding proteins in samples isolated from control individuals and/or with the levels recorded in reference databases, wherein an alteration, in terms of increase, of the levels of said proteins in the sample is indicative of the presence of oral squamous cell carcinoma in the subject.

In a preferred embodiment, this method is carried out using a qualitative ELISA technique, preferably patient side, preferably with dried or freeze-dried reagents, which are brought back into solution and/or mixed at the time of use, obtaining an immediate qualitative chemocolorimetric, bioluminescent response or digitally, detecting and/or quantifying EGFR, p53, B7H6, PdL1, in the biological sample obtained from the subject.

In a preferred embodiment, this method is carried out using a qualitative ELISA technique, also using for processing a specific automatic processing device capable of detecting and/or quantifying signals of the colorimetric, luminescent or bioluminescent type.

In a preferred embodiment, this method is carried out using a quantitative ELISA technique, using for processing a specific automatic processing device capable of detecting and/or quantifying signals of the colorimetric, luminescent or bioluminescent type, preferably with dried or freeze-dried reagents, which are dissolved and/or mixed at the time of use, obtaining an immediate qualitative chemocolorimetric, bioluminescent or digital response, detecting and/or quantifying EGFR, p53, B7H6, PdL1, in the biological sample obtained from the subject.

In a preferred embodiment, the invention refers to a method that can also be performed in environments outside the laboratory, “patient side” with immediate response of a chemocolorimetric, bioluminescent or digital reader nature for the diagnosis and/or for the prediction of the risk of developing oral squamous cell carcinoma in a subject, comprising in vitro detection in a sample isolated from said subject of the markers EGFR, p53, B7H6, PdL1, using an ELISA assay.

The invention also refers to the related kit for diagnosing and/or predicting the risk of developing oral squamous cell carcinoma. The kit according to the invention uses reagents stable at room temperature, is fast, sensitive, specific, portable, inexpensive and non-invasive. In particular, the kit of the invention allows the determination of the markers EGFR, p53, B7H6,PdL1, through a double use of primary antibodies to unequivocally guarantee the specificity and sensitivity of the signal. In particular, a first series of rabbit polyclonal primary antibodies directed against said markers, adhered on a PVDF membrane, a second series of mouse monoclonal primary antibodies directed against said markers, will be used in test tubes. Finally, a third series of secondary anti-mouse antibodies conjugated with an enzymatic signal amplification system, in particular alkaline phosphatase or peroxidase, are used.

The object of the present invention is therefore a disposable kit for testing the presence of the 4 antigens comprising a base having at least a first series and a second series of recesses which can be pierced below and a cover having at least a third and fourth series of recesses which can be pierced above, the position of said recesses being such that the first and second sets of recesses accommodate the third and fourth series of recesses when the base is covered by the cover, and wherein the first or third series of recesses contain first selective biological molecules, preferably antibodies, for selecting said antigens and catalyst molecules bound to the selective biological molecules, such as an HRP enzyme; and the second and fourth series of recesses contain corresponding first and second series of precursors of a chemical reaction, for instance hydrogen peroxide and luminol, which, catalyzed by said catalyst molecules, generates a bioluminescent effect; the kit further comprising a membrane whereon a series of second selective biological molecules are located for selecting and anchoring said antigen to the membrane.

In one embodiment, the kit comprises at least 4 different lines of analysis, one for each of the 4 markers, with side-by-side and preferably aligned sequences of recesses. In a preferred embodiment, positive and/or negative control lines are also included.

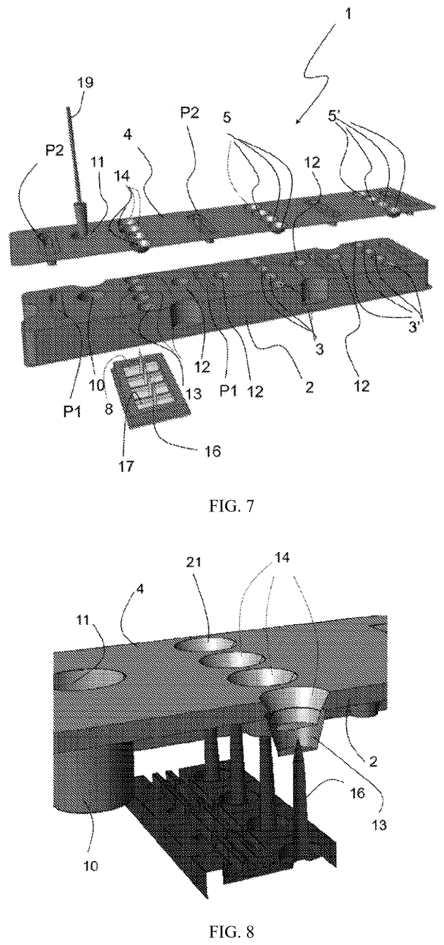

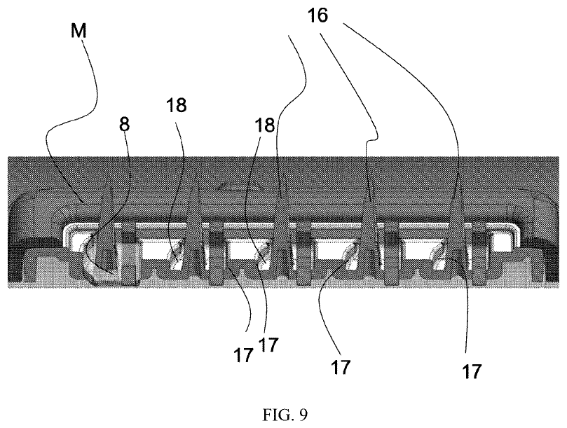

FIG. 7 shows, for the sole purpose of facilitating its interpretation, a kit comprising 4lines (the control line is not illustrated since the perspective view is longitudinally sectioned). The kit comprises a base 2 having a first and a second plurality of recesses 3, 3′, each plurality relating to a stage of the test for the presence of the markers, a cover 4 having a third and fourth plurality of recesses 5, 5′, corresponding shaped coupling portions P1, P2 being provided on the base 2 and on the cover 4 to mount the cover on the base in a unique way and positioned so that the first and second recesses 3, 3′ are housed in the corresponding third and fourth recesses 5, 5′. Consequently, also the third and fourth recesses are correspondingly associated with a corresponding stage of the test for the presence of the markers, the same as the first and second plurality of recesses 3, 3′.

Preferably, base 2 and/or cover 4 are made to keep inside all or some of the substances necessary for the test, e.g. through one or more peelable or hand-drillable layers designed to close and/or seal one or more recesses. Before carrying out the test, these layers are completely or partially removed e.g. by hand or by means of a piercer, so that the liquids in the cover 4 fall by gravity into the recesses of the base 2. The peelable layer/s are e.g. in a polymeric material, in a metallic material or in a combination thereof.

Preferably, in the illustrated embodiment, the molded coupling portions P1, P2 are a recess defined for instance on the base 2 and a corresponding bulge defined on the cover 4 but the reverse is also possible.

Furthermore, in order to reduce production costs and at the same time to maintain high standards of chemical inertness between the reactants and the recesses 3,3′, 5, 5′ which contain them, the base 2 and the cover 4 are made of a plastic material e.g. a polystyrene film, preferably thermo-molded to obtain the desired geometry. As illustrated in the drawings, base 2 and cover 4 made of thermo-molded film define a test tray as a whole. It is preferable that the plastic material of cover 4 is hydrophobic, to allow the complete fall of the liquid reagents into the recesses of base 2 which contain the corresponding powder reagent.

According to the invention, at least a couple of first and third recesses 3, 5 as a whole accommodate a selective biological molecule, for instance an antibody, which selectively interacts with a target antigen such as to bind even to several parts of the same antigen; and a molecule, e.g. biological as an enzyme HRP peroxidase, catalyst of a chemical reaction that generates a bioluminescent substance bound to the selective biological molecule. Furthermore, another couple of second and fourth recesses 3′, 5′ accommodate precursors necessary for the generation of a chemical reaction having a bioluminescent effect and catalyzed by the catalyst molecule. For instance, the precursors of the substance that generates the bioluminescent effect are hydrogen peroxide and 5-amino-2,3-dihydro-1,4-phthalazindione, i.e. luminol. Preferably, in order to increase the efficacy of the biolominescent effect after a period of storage at room temperature, at least one of the precursors e.g. the organic precursor such as luminol is biphasic and stored in a biphasic state on board the kit. The stages join following the breaking or drilling of special recesses on base 2 or on cover 4.

In this way, the contents of the first and third recesses 3, 5 and those of the second and fourth recesses 3′, 5′ are both such as to select and couple with a target antigen and such as to produce a chemical reaction to generate a substance with bioluminescent effect bound to the selected antigen. In particular, the catalyst molecule is bound to the selective biological molecule so that the two have the same localization e.g. within a liquid. The substances of the base 2 and cover 4 interact during the test with a functionalized membrane, whereon the substances fall by gravity as will be better specified hereinafter. Furthermore, in order to preserve the molecules for long periods and to maintain for as long as possible the selecting action of the antigen and the binding which allows to localize the substance which generates the bioluminescent effect, the contents of the first and third recess 3, 5 is bi-phasic e.g. the selective biological molecule and the precursor molecule bound to it are dried and a liquid suspension mixture preferably of emulsifiers and saline buffers is provided, e.g. emulsifiers based on polysorbates and phosphate saline buffers. Furthermore, the second and fourth recesses 3′, 5′ identify two compartments for accommodating the corresponding precursors.

Therefore, the bottom of the third and fourth recesses 5, 5′ is weakened or drillable by hand using a pointed tool (not shown) e.g. of plastic material; in this way it is possible to drop by gravity a dosed, in particular pre-dosed, quantity of substance contained in the corresponding first and second recesses 3, 3′ below to obtain the desired mixtures.

Furthermore, after the first and second recesses 3, 3′ are filled, the functionalised membrane previously exposed to the biological material comprising the antigen is arranged sequentially under each of them, to favor the selection and anchoring of the latter by the antibodies.

Preferably, the bioluminescent effect obtained on the membrane can be captured by an image sensor and subsequently processed by an imaging algorithm e.g. to separate via binarization a background (dark) to whose pixels a background label is associated from a pattern (brighter) obtained through the bioluminescent effect to whose pixels an activation label is associated and count the activated pixels of the pattern to obtain a quantitative indication of antibody-coupled antigens.

Furthermore, according to a preferred embodiment, the kit comprises a reference membrane 8 wherein third selective biological molecules not indicated for the antigen of interest are present. The corresponding recesses, on the other hand, contain the substances of the recesses corresponding to the test membranes. In this way, the kit comprises a substance which allows to generate, after the chemical reaction of the precursors, a substance which generates a bioluminescent effect of comparison whose image allows to define a term of comparison for the images relating to substances with bioluminescent effects generated in the presence of the antigen and useful, as will be explained in greater detail below, for performing image filtering algorithms.

Furthermore, it is possible that the kit comprises initial recesses 10, 11 on the corresponding base 2 and cover 4, overall containing starting substances of the biological test sample containing the target antigen, for instance biological molecules from an oropharyngeal swab. For instance, the starting substances comprise a dried protease inhibitor contained in one of the initial recesses and a lysis solution preferably based on saline buffers. The initial recess 10 can also be drilled to allow mixing of the two substances after drilling and preferably, the lysis mixture is arranged above the protease inhibitor.

Preferably, for the purpose of washing molecules not bound to the antigens, base 2 comprises washing wells 12 containing a substance capable of removing any non-specific bonds on the membranes by the substances coming from the recesses and not bound to the antigens. For instance, the washing substance comprises emulsifiers and saline buffers, e.g. emulsifiers based on polysorbates and phosphate saline buffers, wherein the percentage of saline buffers is greater than that in the suspension solution. For instance, the percentage of saline buffers is 1% in the washing substance and is 0.1% in the suspension substance.

According to a preferred embodiment, the disposable kit 1 is configured to perform an ELISA procedure in sequence and further comprises at least a first and a second recess of primary antibody, preferably monoclonal, 13, 14 on the corresponding base 2 and cover 4 containing a dried antibody, e.g. in recess 13, and a suspension solution e.g. in recess 14. Preferably at least one washing well 12, in the example of FIG. 1 two wells 12, are interposed between the primary antibody recesses 13, 14 and the first and third recesses 3, 5. Furthermore, in the first or third recess 3, 5 primary antibodies are contained. In addition, the substance collecting membrane of the recesses carries antigen-specific antibodies of the corresponding primary monoclonal antibodies. These antibodies are in particular polyclonal. Preferably at least one washing well 12, in the example of FIG. 1 two wells 12, are interposed between the first and third recesses 3, 5 and the second and fourth recesses 3′, 5′. Therefore, as illustrated in the drawing, base 2 and cover 4 have an elongated shape so as to be able to arrange the recesses and wells in longitudinal sequence as described above. This also makes it possible to optimize space when automatic processing machines capable of processing several disposable kits 1 in parallel are provided.

According to the embodiment shown in the figure, the initialization recesses 10, 11 are arranged in an initial position of the sequence, which develops longitudinally along the disposable kit 1. According to a preferred embodiment of the present invention, at least one disposable kit 1 is used in a machine comprising a longitudinally movable shuttle and provided with a lower piercer 16 arranged in use under base 2 and having a material stiffness and/or shape such as to drill (FIG. 8), when the shuttle reaches the suitable position, a corresponding bottom of the recesses 3, 3′, 10, 13 and of the wells 12, 10 in order to make the dosed liquid, in particular pre-dosed, contained in each recess or well, flow out by gravity.

Preferably, the piercer has an elongated shape, e.g. vertical, so as to guide the liquid present in the recesses and/or wells downwards by surface tension before drilling by said piercer.

Furthermore, the shuttle is shaped and comprises a bowl 17 wherefrom the piercer 16 projects and which receives the incoming fluid by gravity. Bowl 17 comprises e.g. on the bottom or other surface wetted by the measured amount of liquid, membrane 18 e.g. based on Poly VinylideneFluoride or other material used in the sector to support antibodies, whereon the non conjugated antigen-specific polyclonal antibodies are applied in localized positions so as to bind to the antigens present in the initial solution containing the biological test material. According to the example of FIG. 1, the initial solution containing the biological test material, taken by means of a brush or swab or cytobrush 19, is obtained by drilling the first initial recess 11 and mixing by the brush 19 the dried protease inhibitor with the lysis solution. The metered amount of dried inhibitor and lysis solution, e.g. 800 microlitres, falls by gravity into bowl 17 after the drilling performed with a relative upward movement of piercer 16 with respect to base 2.

The shuttle continues drilling in sequence on the opposite side of cover 4 with respect to base 2 for each recess 13 (FIGS. 8), 3, 5 and wells 12 and the various quantities of liquid are all deposited in bowl 17 until the chemical reaction is reached which generates the bioluminescent effect.

According to the embodiment variant illustrated in FIG. 7, on the same disposable kit 1 there are side-by-side and preferably aligned sequences of recesses containing test substances based on primary antibodies different from sequence to sequence and, moreover, there is a sequence of reference recesses which, in the embodiment, have the same secondary antibody as the other sequences and one of the primary antibodies of the other sequences. According to a preferred embodiment, the kit comprises a plurality of 5 recesses 13, with the corresponding preferably monoclonal dried and/or dehydrated primary antibody e.g. specific EGFR antibody, specific p53 and specific B7H6, specific PdL1 and one antibody among the above; and a plurality of 5 recesses 3 with the same dried and/or dehydrated conjugated secondary antibody with the catalyst for each of the primary antibodies. Correspondingly, for instance carried by a mask M with corresponding wells, the shuttle carries four membranes 18 each having preferably polyclonal antigen-specific antibodies in combination with the corresponding primary antibodies and the reference membrane 8 having preferably polyclonal antibodies specific for a different antigen, e.g. the melanoma antigen, from that of the other four membranes, for a total of 5 membranes. The membranes 8, 18 are placed in the wells of the mask M. Furthermore, the shuttle comprises a plurality of piercers 16 aligned with the corresponding plurality of 5 recesses/wells (four recesses with test substances and a fifth recess with reference substances) so that, in a stop position of the shuttle, each piercer 16 is under the corresponding recess 13, 3, 3′ and, during a single relative drilling movement, all said recesses are drilled and the liquid contained comes out by gravity reaching the corresponding bowls 17. In the example of FIG. 7, both the series and the piercers 16 are arranged along corresponding lines parallel to each other and superimposed during the relative drilling movement, e.g. a relative vertical movement. The mask M preferably also carries the piercers 16 so that the shuttle comprises both the mask M, which is wetted by the substances to carry out the test, and a mobile support which is controlled by suitable actuators to perform the required movements in a repetitive and programmed manner to perform the steps up to the detection and capture of the digital image of the wells after the detection.

Preferably, the wells 12 and/or the first initial recess 10 each contain a metered, in particular pre-metered, volume of liquid sufficient to supply suitable quantities of substances into all the bowls 17 of the shuttle and, therefore, during the drilling movement, can be drilled by one or at least two piercers 16. Preferably, wells 12 and the first initial recess 10 are aligned along an axis of symmetry of the series of five recesses 13, 3, 3′ and 14, 5, 5′.

Through the use of a kit structured in this way, it is possible to create a kit that exploits both the advantages of bioluminescence i.e. greater precision and uniqueness of interpretation, particularly in the case of process automation, and the availability of a precise and usable instrument outside the analysis laboratories.

By way of non-limiting example in one embodiment the kit comprises a cytobrush or other instrument intended for collecting the biological material containing cells of the oral cavity. As an alternative to the cytobrush, it is in fact possible to use other aids capable of obtaining a sample of the oral cavity containing cells.

The object of the invention is therefore a kit for the diagnosis and/or prognosis of oral squamous cell carcinoma comprising two series of primary antibodies anti-EGFR, anti-p53, anti-B7H6,anti-PdL1 from two different animal species, preferably mouse and rabbit, and a signal detection and/or quantification system consisting of a secondary antibody, conjugated with alkaline phosphatase or peroxidase, directed against one of the two animal species from which the primary antibodies come and optionally instructions for use.

According to a further embodiment, the object of the present invention is achieved by a machine for processing the disposable kit as indicated above according to the previously described method comprising, a housing for at least one disposable kit, a closing lid movable between an opening for loading/unloading the disposable kit from the housing and a closed position wherein the lid is placed above the kit, the mobile shuttle arranged below said housing to support at least one bowl equipped with said membrane and a piercer for the recesses of the base, a mechanism for moving the shuttle and/or piercer relative to the housing and enabling in use the underside drilling of the base recesses, and an electronic control unit programmed to drive the mechanism according to a predefined sequence of positions of the membrane under the housing.

This machine is compact and allows precise tests to be performed even outside specialized laboratories, such as in pharmacies for instance.

### EXAMPLES

The prospective study was conducted in the following centers: Galliera Hospital (Genoa, Italy) and San Camillo Hospital (Rome, Italy) and Vanvitelli University (Naples, Italy) from October 2022 to December 2022.

All patients who came to the centers with a confirmed diagnosis of oral cancer were examined consecutively, regardless of age, sex, ethnicity, general health conditions.

All patients enrolled in the study were diagnosed with primary OSCC stage I-IV (T1-T4) according to TNM (tumor-node-metastases) criteria without prior chemotherapy or radiation therapy. All protocols used in this study were approved by the Ethics Review Committees, the Institutional Review Committee of the University of Genoa Italy, and all participating institutions were enrolled.

### Sample Collection

A detailed history was collected using a predefined form. Written informed consent was obtained from all patients.

CYTOBRUSH: Samples for analysis were taken with a cytobrush (Meringer SpA).

Patients were asked to rinse their mouths with saline before taking the cytobrush biopsy. Three cytobrush samples were taken from each patient's mouth by fastly rotating the cytobrush 360°, applying sufficient pressure on the brush to collect cells and exfoliated tissue fragments, but limiting bleeding as much as possible. The cytobrush tips were inserted into sealed Eppendorf vials and stored cold until analysis.

Samples deteriorated due to non-compliance with the storage and/or transport protocol, samples with relevant blood fractions, samples with cell fraction below the cut-off threshold (10 femtograms/microliter) were excluded from the analysis.

Three non-invasive cytobrush biopsies were taken from each patient. Each cytobrush was rubbed by applying gentle pressure and rotation over the area to be analyzed. The cytobrush was brushed vigorously, but without causing bleeding at the sampling site. Each cytobrush harvested cells from one of three target regions of the study: tumor center, tumor margin, healthy control tissue.

In each patient, one sample was obtained from the center of the lesion (group A), one from the margin of the lesion (group B), and one from the surrounding healthy tissue (group C). Samples were listed and stored cold at 0-4° C. and sent to the laboratory for analysis in refrigerated boxes.

BIOPSIES: The biopsy fragment (one for tumor center area, one for margin, one for healthy tissue) was received in a closed refrigerated container (0-4 degrees), the tissue was then washed with 1 mL of PBS (SERVA buffer Dulbecco's substance) three times on a rocking shaker (ARGO-LAB SKO-D XL). The fragment was cut to approximately 1 square centimeter in size, inserted into 2 mL Eppendorf to which 800 μL of lysis buffer (EMD millipore RIPA lysis buffer) and protease inhibitor cocktail (SIGMA Protease inhibitor cocktail) were added. The solution was homogenized for ten minutes in an automatic homogenizer (PREOMICS BeatBox) and subsequently centrifuged (Healttrow scientific SPROUT PLUS) at 2000 r for 10 minutes. The supernatant was used for subsequent analysis.

Analysis: The samples were processed with the automated Femtohunter device (Stark sarl principality of Monaco). The Femtohunter is a device that develops a fast quantitative ELISA test with a chemiluminescent result. The system has an analytical sensitivity of 10 femtograms/microliter using kits according to the invention.

Analysis of the samples: The biological samples were inserted into the corresponding cavities of the reagent slot of the Stark kits.

The markers were analyzed following the following order: first analysis slot formed by the markers EGFR, p53, Ki67 plus control membrane and second analysis slot formed by the markers B7H6, PDL1, HLAE, control.

The membrane is a PVDF membrane (Thermofisher scientific catalog number LC2002).

The reagent slots, loaded with the biological sample from the patient, were inserted into the automatic development device with the chemiluminescent response Elisa process.

The slot membranes, loaded with polyclonal antibodies against the marker of interest, were inserted into the automatic development device. The automatic development procedure takes place in 13 development steps, 12 for the ELISA procedure plus one for light signal detection and analysis:
1. Activation of PVDF Membranes
2. Loading the membrane slot with lysed biological sample.
3. Washing of the membranes to remove excess unbound material
4. Addition of BSA solution to block non specific sites on PVDF membrane
5. Washing of membranes to remove unbound proteins
6. Step of loading monoclonal antibodies against markers of interest onto the PVDF membrane to form a sandwich
7. Membrane washing to remove non-specific attachments
8. Membrane washing to remove non-specific attachments
9. Loading of the membrane with HRP-conjugated secondary monoclonal antibody
10. Membrane washing to remove non-specific attachments
11. Membrane washing to remove non-specific attachments
12. Loading step of the membrane with chemiluminescent sensing substrate
13. Detection of the chemiluminescent signal and analysis of the light intensity present on the PVDF membranes at a wavelength of 425 nm

### Antibodies

Polyclonal antibody anti-EGFR protein GENETEX GTX121919; Monoclonal antibody against EGFR protein GENETEX GTX628887; Polyclonal antibody anti AR protein GENETEX GTX100056; Monoclonal antibody anti AR protein ABCAM ab9474; Polyclonal antibody anti ER protein GENETEX GTX127978; Monoclonal antibody anti ER protein GENETEX GTX70171; Polyclonal antibody anti p16R protein ABCAM Ab189034; Monoclonal antibody anti p16 protein ABCAM Ab201980; Polyclonal antibody anti p53 protein ABCAM Ab131442; Monoclonal antibody anti p53 protein ABCAM PAb1801; Polyclonal antibody anti Ki67 protein ABCAM Ab15580; Monoclonal antibody anti Ki67 protein INVITROGEN MA5-15690; Polyclonal antibody anti PDL1 protein ABCAM Ab233482; Monoclonal antibody anti PDL1 protein R&D SYSTEMS MAB1561R; Polyclonal antibody anti B7H6 protein ABCAM ab229999; Monoclonal antibody anti B7H6 protein R&D SYSTEMS MAB7144; Polyclonal antibody anti HLAE protein ABCAM Ab203082; Monoclonal antibody anti HLAE protein Antibodies. com A121801.

### Statistic Analysis

All statistical analyzes were performed using R 2.9.1 statistical software and Bioconductor 2.5 (packages: ROC, meta). Averages and standard deviations were calculated for samples from OSCC Test Groups A, B and Control Group C for each marker. The differential expression of each marker was assessed within each cohort using the Mann-Whitney U test to determine statistical significance and by constructing a receiver operating characteristic curve (ROC). The area under the curve (AUC) of each of the ROC curves was obtained by numerical integration. Sensitivity and specificity were evaluated for each marker in each group, using an expression cut-point corresponding to the pth percentile value for the marker, where p is the proportion of OSCC cases in the cohort under examination.

For the meta-analysis of individual markers, estimates of sensitivity and specificity were used to generate a pooled estimate using fixed and random effects inverse variance meta-analysis models. These models were constructed separately for sensitivity and specificity and were weighted proportionally to the relevant sample sizes of each study. The sensitivities and specificities of individual cohorts were used instead of the original marker values, due to differences in measurement scales between experiments and differences in the proportions of OSCC cases in each cohort. We also performed a meta-analysis to compare marker expression between cancer and controls. This analysis used a within-group z-transform to normalize expression levels between cohorts and then used a mixed-effects model to compare z-transformed values between cancer and controls. For classification model evaluation, logistic regression was used to examine the utility of marker combinations for classification of OSCC. Two models were built for each cohort. First, the same markers as in the original report were used in each group. Then, stepwise advanced regression within each group was used to identify the top three markers for each group. The AUC was calculated by constructing a ROC curve using the probabilities predicted by each logistic model and then numerically integrating the curve. The sensitivity and specificity of the models were calculated in the same way as for the individual marker models.

1. An in vitro method for the diagnosis and/or prognosis of oral squamous cell carcinoma comprising the following steps:
providing an isolated sample from an individual,
analyzing protein levels of EGFR, p53, B7H6, PdL1, in the sample,
comparing the protein levels in the sample with levels of corresponding proteins in samples isolated from control individuals and/or with levels recorded in reference databases,
wherein an alteration, in terms of increase, of the levels of the proteins in the sample is indicative of a presence of oral squamous cell carcinoma in the individual.

2. The method according to claim 1 wherein the protein level analysis step is performed with an ELISA technique.

3. The method according to claim 1 wherein the sample is isolated from a buccal swab, a cytobrush smear, saliva or any sample containing oral cavity epithelial cells or from a biopsy comprising oral cavity epithelial tissue.

4. The method according to claim 1, wherein the step of analyzing further comprises detecting via bioluminescence, and further comprises detecting a digital image of the bioluminescent detection and processing, preferably binarizing, the image to associate each image pixel with a background label corresponding to lower brightness or an activated pixel label representative of a bioluminescent effect and corresponding to greater brightness.

5. A kit for diagnosis and/or prognosis of oral squamous cell carcinoma comprising:
a device suitable for taking a sample of an oral cavity containing cells, preferably a cytobrush,
two series of primary antibodies, preferably one series of monoclonals and one series of polyclonals, anti-EGFR, anti-p53, anti-B7H6, anti-PdL1 E′ MEGLIO ANTI o AGAINST? from two different animal species,
a signal detection and/or quantification system comprising a secondary antibody, conjugated with a detection compound, preferably alkaline phosphatase or peroxidase, directed against one of two animal species from which the primary antibodies come
and optionally instructions for use.

6. The kit according to claim 5, wherein the kit is disposable and the two sets of primary antibodies are one immobilized on a support, e.g. a solid support of PVDF, and the other dried/freeze-dried to be solubilized at the time of use.

7. The disposable kit according to claim 6, comprising a base having a first plurality of recesses for one of the series of primary antibodies; and a cover having a second plurality of recesses for a primary antibody solubilization liquid, the cover being configured so that, when superimposed on the base, the second plurality of recesses is housed in the first plurality of recesses and at least the second plurality of recesses can be pierceable by hand using a tool.

8. The disposable kit according to claim 7, wherein at least one recess is closed or sealed at the top before use by means of a peelable or pierceable layer.

9. The disposable kit according to claim 6, further comprising a disposable mask (M) comprising at least one recess whereto the other of the two sets of primary antibodies is fixed.

10. A method of using the kit according to claim 5 in an in vitro method for the diagnosis and/or prognosis of an oral squamous cell carcinoma.

11. The method according to claim 2 wherein the sample is isolated from a buccal swab, a cytobrush smear, saliva or any sample containing oral cavity epithelial cells or from a biopsy comprising oral cavity epithelial tissue.

12. The method according to claim 2, wherein the step of analyzing further comprises detecting via bioluminescence, and further comprises detecting a digital image of the bioluminescent detection and processing, preferably binarizing, the image to associate each image pixel with a background label corresponding to lower brightness or an activated pixel label representative of a bioluminescent effect and corresponding to greater brightness.

13. The method according to claim 3, wherein the step of analyzing further comprises detecting via bioluminescence, and further comprises detecting a digital image of the bioluminescent detection and processing, preferably binarizing, the image to associate each image pixel with a background label corresponding to lower brightness or an activated pixel label representative of a bioluminescent effect and corresponding to greater brightness.

14. The disposable kit according to claim 8, further comprising a disposable mask (M) comprising at least one recess whereto the other of the two sets of primary antibodies is fixed.
